(19) United States
(12) Patent Application Publication
(10) Pub. No.: US 2011/0059441 A1
(43) Pub. Date: Mar. 10, 2011

(54) METHOF OF PRODUCING BIOACTIVE PAPER

(75) Inventors: Robert Pelton, Dundas (CA); Shunxing Su, Hamilton (CA); Carlos Filipe, Ancaster (CA); Yingfu Li, Dundas (CA)

(73) Assignee: MCMASTER UNIVERSITY, Hamilton, ON (CA)

(21) Appl. No.: 12/595,857

(22) PCT Filed: Apr. 16, 2008
(86) PCT No.: PCT/CA08/00696

§ 371 Date: Nov. 24, 2010

Related U.S. Application Data
(60) Provisional application No. 60/912,028, filed on Apr. 16, 2007.

(51) Int. Cl. C12Q 1/68; B05D 5/00; B05D 3/02; G01N 21/76; G01N 21/75; G01N 33/53; G01N 30/96; C12M 1/34

(52) U.S. Cl. 435/6; 427/288; 427/372.2; 436/172; 436/164; 435/7.1; 422/69; 435/287.2; 435/287.9

(57) ABSTRACT

The present disclosure relates to methods for attaching bioactive agents to paper products by contacting the paper with a solution comprising colloidal support particles where said colloidal support particles are associated with bioactive agents. In specific embodiment of the disclosure, the colloidal support particles are functionalized poly(N-isopropylacrylamide) microgels. The disclosure further covers the bioactive paper produced by this method as well as uses thereof, in particular for pathogen detection.

The present disclosure relates to methods of attaching bioagents to paper and paper products, to the paper and paper products prepared using this method as well as various uses of these products, in particular for pathogen detection.

### BACKGROUND

Over the past hundred years, paper-based food packaging, face masks and protective clothing have played an important role in protecting us from pathogens. These applications of paper reflect the fact that it is inexpensive, disposable, sterile and can have well defined porosity. Nevertheless, in most protective applications, paper functions simply as a passive barrier or filter. Paper has also been utilized as the substrate for developing chromatographies to purify samples, such as amino acids, nucleotides, or proteins (Paintanida, M.; Meniga, A.; Muic, N., A contribution to paper-strip chromatography of proteins. Archives of biochemistry and biophysics 1955, 57, (2), 334-9; Rubery, E. D.; Newton, A. A., Simple paper chromatographic method for separation of methylated adenines and cytosine from the major bases found in nucleic acids. Analytical Biochemistry 1971, 42, (1), 149-54; McFarren, E. F., Buffered filter paper chromatography of the amino acids. Anal. Chem. 1951, 23, 168-74). Chromatographic migration of DNA on a nitrocellulose strip has also been utilized to detect very tiny amount of target virus DNA in a short time (Reinhartz, A.; Alajem, S.; Samson, A.; Herzberg, M., A Novel Rapid Hybridization Technique-Paper-Chromatography Hybridization Assay (Pacha). Gene 1993, 136, (1-2), 221-226). A paper-supported biosensor which exploited the chromatographic properties of paper was recently described (Martinez, A. W.; Phillips, S. T.; Butte, M. J.; Whitesides, G. M. Angewandte Chemie-International Edition 2007, 46, 1-4).

A survey of the patent and literature for bioactive paper and fibre products was recently published (Aikio, S. et al., Bioactive paper and fibre products: Patent and literary survey, VTT Working Papers, Julkaisija—Utgivare Publisher, 2006, ISBN 951-38-6603-3).

Previous work has demonstrated the ability to covalently couple ATP DNA aptamers onto regenerated cellulose membranes with retention of their activities (Su, S.; Nutiu, R.; Filipe, C. D. M.; Li, Y.; Pelton, R. Langmuir 2007, 23, (3), 1300-1302).

### SUMMARY OF THE DISCLOSURE

Bio-recognition molecules have been attached to colloidal microgel particles and these particles have been formulated into inks and coatings and applied to paper products. It has been shown that the attached molecules retain their bio-recognition properties when applied to the paper.

Accordingly, the present disclosure relates to a method for attaching bioactive agents to paper products comprising contacting the paper with a solution comprising colloidal support particles under conditions for the immobilization of the particles to the paper, where the bioactive agents are immobilized on the colloidal support particles.

In an embodiment of the present disclosure, the colloidal support particles are poly(N-alkylacrylamide) or poly(N,N-dialkylacrylamide) microgels optionally comprising functional groups at or near their surface.

The present invention further comprises the paper products comprising bioactive agents associated therewith as well as the use of these products in, for example, bio-recognition and bioseparation applications.

The present research finds applications, for example, in the development of paper-supported biosensors, for uses such as pathogen detection. Many biosensing schemes involve bio-recognition molecules such as enzymes, antibody fragments, DNA aptamers and the like. Generally, such molecules are expensive and fragile and the must be carefully coupled (covalently bonded) to the support in order to be immobilized while maintaining activity. Paper, while convenient, is a difficult support to use for these applications because it is rough and non-uniform and can have a wide variety of surface chemistries. For example, to function in water, paper must be impregnated with wet-strength resin which is usually a cationic crosslinked polymer which can denature proteins and other sensitive biomolecules. Furthermore, the chemistry for coupling bioactive agents is often sensitive and not compatible with papermaking, printing or coating technologies. This has made the direct application of a wide range of bio-recognition molecules to a wide range of paper substrates by single technology challenging.

The colloidal support particles of the present disclosure offer the following unexpected advantages:
1. The particles adhere strongly to a wide variety of paper surfaces. They neither desorb when immersed in buffer, not do the particles move in a paper chromatography.
2. It has been shown that proteins (for example antibodies) and oligonucleotides (for example DNA aptamers) maintain their activity when coupled to the particles and absorbed onto paper, in particular, cationic paper surfaces.
3. The particles can be applied to paper surfaces by conventional printing and coating technologies.

It is not convenient to couple bioactive agents onto paper surface after the paper is manufactured. Moreover, different chemistries may have to be employed for putting different bioactive agents onto paper surfaces, and these chemical reactions may destroy their activities, especially fragile proteins. In addition, the chemistries of paper surfaces can be very different for various paper products, which must be considered when depositing bioactive agents onto paper surfaces. Thus, a universal platform that is applicable for any bioagent that should not destroy the bioagents' activities after being coupled, such as that disclosed herein, is highly desirable.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is a schematic showing one embodiment of microgel derivation.

FIG. 2A is a schematic showing two embodiments for applying bioactive paper for pathogen detection.

FIG. 2B is a schematic showing an embodiment for sample preparation and paper chromatography experiments.

FIG. 3 is a graph showing the pH dependence of the electrophoretic mobility of poly(NIPAM)-VAA microgels (MG) and Rhodamine B-labelled microgel (RB-MG) at 25° C. in 1 mM NaCl.

FIG. 4 shows pictures of filter paper strips (blank and RB-MG labelled) before and after developing in 20 mM sodium phosphate buffer (pH 7.4). The paper strips before and after chromatographies were scanned by Typhoon.

FIG. 5 shows pictures of filter paper strips labelled with RB-MG that have been washed in buffer (20 mM sodium phosphate, 300 mM NaCl, 0.1% Tween 20). The strips were either pretreated with (1) PAE, (2) PAE then CMC or (3) PAE then PAA. The paper strips before and after washing were scanned by Typhoon.

FIG. 6 is a confocal image showing N optical cross-section band of RB-MG microgel spotted on the filter paper shown in FIG. 4.

FIG. 7 shows pictures of filter paper strips on to which DNA oligo (0.5 μl, 10.5 μM) or BSA (2 μl, 0.72 mg/mL) was dropped right below the microgel region. Chromatographies were done in sodium phosphate buffer (20 mM, pH 7.4) and the paper stripes were scanned by Typhoon.

FIG. 8 is a graph showing the pH dependence of the microgel's size (measured at 25° C.). The measurement were made in 0.001 M NaCl. The error bars denote three replicates.

FIG. 9 is a graph showing the pH dependence of the microgel's electrophoretic mobility at 25° C. in 0.001 M NaCl. The error bars represent 10 runs (15 cycles each).

FIG. 10 shows a schematic diagram of the ATP-aptamer recognition of adenosine triphosphate (ATP) structure-switching signaling aptamer. Fluorescent intensity decreases upon duplex formation; fluorescence increased when ATP binding disrupted the duplex. The graph in this figure demonstrates that the microgel supported aptamer, ATP-MG, retains its ability to recognize ATP and not guanosine triphosphate (GTP). Measurements were made in the binding buffer (300 mM NaCl, 5 mM MgCl2, 25 mM Tris-HCl, pH) 8.3). The upper line was displaced by 20 units in the y axis.

FIG. 10 shows pictures illustrating the APT-MG activity on filter paper. The strips were eluted with ATP or GTP in binding buffer. The darker regions correspond in the monochrome image correspond to higher fluorescence. Microgel concentration: 6.5 mg/mL.

FIG. 11 shows pictures of test paper strips illustrating ATP detection by microgel supported DNA-aptamers spotted (left) and printed onto unmodified filter paper surfaces. The ink-jet samples were eluted with 2 mM ATP or GTP in binding buffer.

FIG. 12 shows pictures of test paper strips illustrating a comparison of DNA aptamer, directly applied, with APT microgel on paper treated with 0.1% cationic PAE solution.

FIG. 13 shows pictures of test paper strips that illustrate the activity of IgG-MG using procedure 1 in Table 1. The IgG-MG and IgG-MG-control concentrations were 10 mg/mL. The Ag-Per and Per concentrations were 0.08 mg/mL.

FIG. 14 shows pictures of test paper strips that illustrate the activity of IgG-MG microgels on paper using procedure 2 in Table 1. The microgel concentrations were 10 mg/mL. The Ag-Per and Per concentrations were 1.6 μg/mL

### DETAILED DESCRIPTION OF THE DISCLOSURE

### Definitions

The term “paper” and “paper products” as used herein refers to a commodity of thin material produced by the amalgamation of fibers, typically vegetable fibers composed of cellulose, which are subsequently held together by hydrogen bonding. While the fibers used are usually natural in origin, a wide variety of synthetic fibers, such as polypropylene and polyethylene, may be incorporated into paper as a way of imparting desirable physical properties. The most common source of these kinds of fibers is wood pulp from pulpwood trees. Other vegetable fiber materials, including those of cotton, hemp, linen and rice, may also be used.

The term “microgel” as used herein refers any colloidally stable, water-swellable polymeric network particle whose diameter typically ranges from about 50 nm to about 5 μm.

The term “immobilized” as used herein means to affix a first entity to a second entity such that, under conditions of normal use (i.e. the use for which it was intended), the first and second entities remain substantially affixed. The immobization may be by any means, including physical attachment (e.g. covalent bonding) or attractive forces (e.g., hydrogen bonding, ionic interactions).

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### Method of Producing Bioactive Paper and Uses Thereof

As a first step for the development of biosensing inks for packaging and other paper-based applications, carboxylic poly(N-isopropylacrylamide) microgels with covalently coupled antibodies (anti-mouse) or DNA aptamers (ATP structure-switching signaling) were printed on paper surfaces while maintaining recognition capabilities. The microgels were stationary during chromatographic elution and there was sufficient transport of soluble substrate during elution to the microgel supported antibodies or aptamers to give visible signals.

Accordingly, the present disclosure relates to a method for attaching bioactive agents to paper products comprising contacting the paper with a solution comprising colloidal support particles under conditions for the immobilization of the particles to the paper, where the bioactive agents are immobilized on the colloidal support particles.

In an embodiment of the present disclosure, the colloidal support particles are made from any material that forms temperature-sensitive microgel particles, that does not negatively affect the activity of the bioactive agent and that will irreversibly attach to paper and paper products. Examples of such particles include microgels prepared from starch, cross-linked poly(sodium methylacrylate), poly(N-acryloylpyrrolidine), poly(N-acryloylpiperidine), poly(N-vinylisobutyramide), gums, functionalized latex, agarose and functionalized poly(N-alkylacrylamides) or poly(N,N-dialkylacrylamides). The colloid support particles further include particles having a microgel shell and a core comprising any other material including, for example, hydrophobic polymers, magnetic particles and inorganic nanoparticles. Such core/shell particles are known in the art (see, for example, Pichot, C.; Taniguchi, T.; Delair, T. Elaissari A. Journal of Dispersion Science and Technology 2003, 24(3-4), 423-437.

In an embodiment of the present disclosure, the colloidal support particles are carboxylated poly(N-alkylacrylamide) or poly(N,N-dialkylacrylamide) microgels. In a further embodiment, the N-alkylacrylamide or N,N-dialkylacrylamide is selected from N-isopropylacrylamide, N-ethylmethylacrylamide, N-n-propylacrylamide, N-methyl-N-n-propylacrylamide, N-isopropylmethylacrylamide, N-ethylacrylamide, N,N-diethylacrylamide, N-n-propylmethylacrylamide, N-cyclopropylacrylamide and N-methylacrylamide, in particular N-isopropylacrylamide.

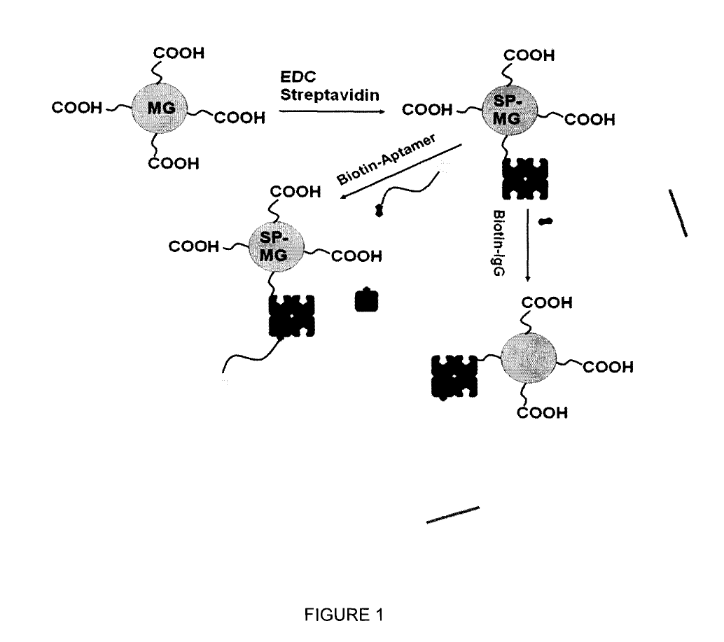

In an embodiment of the present disclosure, the colloidal support particles comprise a functional group at or near their surface for immobilization of bioactive agents. Means for immobilizing bioagents on the colloidal support molecules are known to a person skilled in the art. For example, in an embodiment of the present disclosure the bioactive agents are immobilized on the colloidal support particles by a covalent attachment with a carboxyl, amino, thiol, aldehyde, cyano, hydroxyl, tosyl or hydrazine group, suitably a carboxyl or amino group, located at or near the surface of the particles (see FIG. 1). As a representative example, carboxyl groups may be located at the surface region of poly(N-alkylacrylamides) or poly(N,N-dialkylakylamides) by copolymerization with vinyl acetic acid (VAA). Specific examples of this include carboxylated poly(N-isopropylacrylamide) microgels prepared by copolymerization of N-isopropylacrylamide with vinyl acetic acid (VAA) (Hoare, T.; Pelton, R. Macromolecules 2004, 37, 2544-2550; Hoare, T.; Pelton, R. Langmuir 2004, 20, 2123-2133), amine containing microgels prepared by copolymerization of N-isopropylacrylamide with N-vinylformamide (Xu, J. J.; Timmons. A. B.; Pelton, R. Colloid and Polymer Science 2004, 282(3), 256-63) and thiol containing microgels prepared by copolymerization of N-isopropylacrylamide with vinylbenzylisothiouronium chloride (Meunierm F.; Elaissari, A.; Mallet, F.; Pichot C. Langmuir 2000, 16(23), 9002-9008). Other carboxylic monomers that may be used to incorporate carboxyl groups into the colloidal support particles include, for example, acrylic acid, methacrylic acid, fumaric acid and maleic acid (Hoare, T.; Pelton, R. Journal of Colloid and Interface Science 2006, 303(1), 109-116).

It is to be understood that the bioactive agents may be immobilized on the colloidal support particles before or after contacting the particles to the paper. In an embodiment the bioactive agents are immobilized on the colloidal support particles before contacting the particles to the paper.

The present disclosure relates to methods for the preparation of bioactive paper and accordingly, the term “bioactive agent” will typically refer to any type of bio-recognition molecule. Such molecules include, for example, any proteins, polypeptides, polynucleotides (DNA or RNA), nucleotide fragments, carbohydrates, other polymeric species, cage compounds and small inorganic or organic molecules. Some specific examples of bioactive agents include, for example, antibodies, antibody fragments, probes, primers, enzymes, catalysts, drugs, chelating agents and biotin. A person skilled in the art would appreciate that the bioactive agent need not useful for “bio-recognition” but can be useful for other applications, such as drug delivery. Further, more than one type of bioactive agent may be associated with the colloidal support particles.

In an embodiment of the present disclosure, the contacting of the paper with a solution comprising the colloidal support particles under conditions for the immobilization of the microgels to the paper is done using a micropipette. Alternatively, the colloidal support particles are formulated as an ink and are deposited on the paper using any printing technique known in the art. For a review of the printing techniques that may be applied to bioactive paper and that are known in the art, see Aikio, S. et al., Bioactive paper and fibre products: Patent and literary survey, VTT Working Papers, Julkaisija—Utgivare Publisher, 2006, ISBN 951-38-6603-3. Further, the conditions for the immobilization of the microgels to the paper also comprise drying the paper after contacting with the microgel solution. In an embodiment, the drying is done by allowing the paper to sit in air for a suitable amount of time. The time required for drying the paper comprising the microgel solution deposited thereon would depend on the identity of the solvent and atmospheric conditions, such as temperature, humidity and pressure, but would none-the less be determinable by a person skilled in the art.

In a further embodiment of the present disclosure, the paper is treated prior to contact with the microgel solution, for example, to minimize non-specific binding, to increase the paper wet strength or to neutralize charges on the paper or other pre-treatment. Such methods of treating paper for chromatographic applications are well known to those skilled in the art.

The present invention further comprises the paper products comprising bioactive agents associated therewith as well as the use of these products in biorecognition, bioseparation and other applications.

Accordingly, the present disclosure further includes a method of detecting a target substance comprising contacting a solution or gas suspected of containing the substance with the bioactive paper or paper product of the present disclosure and observing a detectable change in an area on the paper where a bioactive agent has been deposited.

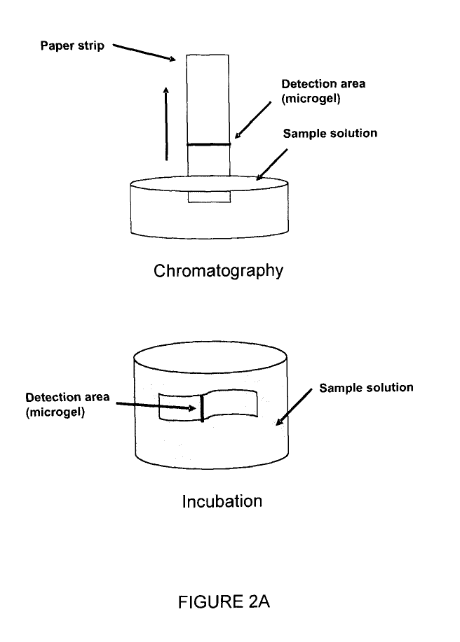

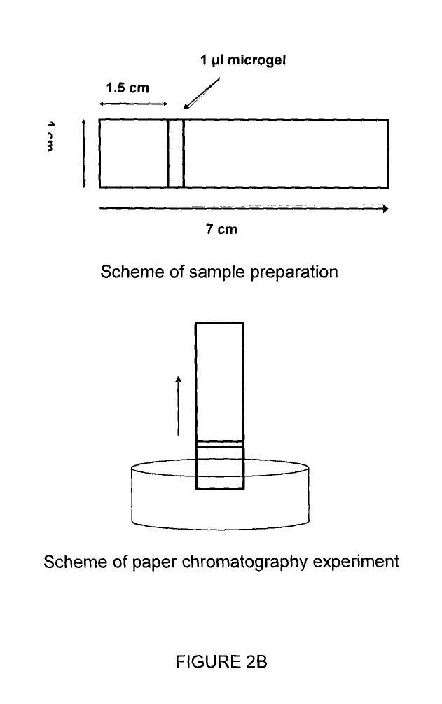

FIGS. 2A and 2B shows two embodiments of the present disclosure for the application of the particles described herein in bioactive paper detection applications. One is to deposit the bioactive agents on the paper surface and then either put the sample suspected of containing the substance to be detected on the paper strip or put it in the developing buffer. After a paper chromatography, a color or fluorescence change should be detected in the detection area for a positive result. The other is to deposit the bioactive agents on the paper and then do an incubation experiment to get a signal in the detection area. To detect the interaction between the bioactive agent and the substance to be detected, an observable change in the “detection” area on the paper occurs as a result of the interaction between the bioactive agent and the substance. The “detection area” refers to that area on the paper or paper product where the colloidal support particles have been deposited. Such detection methods are known to those skilled in the art and may include, for example, a detectable change in the color, fluorescence, ultraviolet or infrared properties of the bioactive agent and/or substance.

The bioactive paper products may also be used in any type of chromatographic application, for example to separate and/or isolate a desired or undesired substance from a mixture. The present disclosure therefore also includes a method of performing a chromatographic separation of one or more components of a mixture comprising, applying the mixture to the bioactive paper or paper product of the present disclosure and performing a chromatographic separation of the components of the mixture. Methods for the separation of components of mixtures using paper chromatography are well known in the art.

The substance may be any molecular species, cell or organism which one wishes to detect or isolate. In one aspect of the present disclosure, the substance is a pathogen or a toxic substance.

The following non-limiting examples are illustrative of the present invention:

### EXAMPLES

### Example 1

### Deposition of Poly(NIPAM) Microgels on to Paper (A) Materials

N-Isopropylacrylamide (NIPAM, 99%, Acros Organics) was purified by recrystallization from a 60:40 toluene/hexane mixture. N,N-Methylenebisacrylamide (MBA), vinylacetic acid (VAA, 97%), sodium dodecyl sulfate (SDS), 2-(N-morpholino)ethanesulfonic acid (MES), adenosine 5′-triphosphate (ATP), guanosine 5′-triphosphate (GTP), carboxymethyl cellulose (CMC), polyacrylic acid (PAA) and ammonium persulfate (APS, 99%) were all from Sigma Aldrich and used as received. The water used in the synthesis was Milli-Q water. Lissamine rhodamine B ethylenediamine, fluorescein isothiocynate (FITC), and HPLC purified DNA oligonucleotide (5′ fluorescein-TCGACTAAGCACCTGTCTTCGCCTT 3′ [SEQ ID NO: 1]) were from Invitrogen. The oligonucleotide was diluted to a final concentration of 10.5 μM using Milli-Q water. N-Ethyl-N′-(3-dimethylaminopropyl)carbodiimide hydrochloride (EDC), N-hydroxysuccinimide (NHS), bovine serum albumin, streptavidin (SP, MW ˜60 kDa), peroxidase, o-phenylenediamine dihydrochloride (OPD), anti-mouse IgG (whole molecule) peroxidase conjugate MW 44 kDa, and anti-rabbit IgG (whole molecule) biotin conjugate were from Sigma. Polyamideamine-epichlorohydrin (PAE) resin was provided by Hercules, Inc. (Kymene 557H). Fluorescein-C6-5′TCACTGACCTGGGGGAGTATTGCGGAGGAAGGTTTT3′-C6-Biotin [SEQ ID NO: 2] (FDNA, MW 12.2 kDa) and 4-(4-dimethylaminophenylazo)benzoic acid (DABCYL)-3′GTGACTGGACCC [SEQ ID NO:3] (QDNA, MW 4.1 kDa) were from Integrated DNA Technologies, with HPLC purification.

All protein concentrations were measured by Bradford (Sigma) microassay with a UV-vis spectrophotometer (Beckman Coulter, DU 800). Fluorescence intensity measurements were performed with a Cary Eclipse fluorescence spectrophotometer (Varian), using an excitation wavelength of 490 nm and an emission wavelength of 520 nm.

### Example 1

### Microgel Preparation

The polyNIPAM microgel with carboxyl groups on the exterior layer was prepared as described in the literature (Hoare, T.; Pelton, R., Highly pH and temperature responsive microgels functionalized with vinylacetic acid. Macromolecules 2004, 37, (7), 2544-2550; Hoare, T.; Pelton, R. Langmuir 2004, 20, 2123-2133). Briefly, emulsion polymerization was performed in a 500 mL three-necked flask, which was assembled with a condenser and a glass stirring rod with a Teflon paddle. A 1.48×10⁻² mol portion of NIPAM, 7.8×10⁻⁴ mol of MBA, 2.0×10⁻⁴ mol of SDS, and 1.48×10⁻³ mol of VAA were all dissolved in 220 mL water and bubbled with nitrogen for 30 mins. APS (5.2×10⁻⁴ mol) was dissolved in 10 mL of water and injected to the flask. The flask was then incubated at 70° C. to start the reaction and the polymerization was carried out overnight with 200 rpm stirring. After cooling, all micro gels were purified by several cycles of ultracentrifugation (Beckman model L7-55, 50 min at 50,000 rpm), decantation, and redispersion in Milli-Q water until the supernatant conductivity was less than 5 μS/cm. The microgel was lyophilized and stored at room temperature. The carboxyl group content of the polyNIPAM-VAA microgel was measured to be 0.248 (0.023 mmol/g by simultaneous conductometric and potentiometric titration with a Burivar-I2 automatic buret (ManTech Associates).

### Example 2

### Coupling of Lissamine Rhodamine B onto the Microgel

The lyophilized microgel was resuspended in sodium phosphate buffer (0.1 M, pH 7.2) at a concentration of 2 mg/mL and incubated overnight before performing the coupling reaction. A 2.5 mL portion of this microgel suspension was reacted for 4 hours and at room temperature with 100 μl of Lissamine Rhodamine B ethylenediamine (2 mg/mL in DMSO), in the presence of 100 mM EDC and 25 mM NHS. A control experiment was done using the same procedure but without EDC and NHS being added. After the reaction, the microgel was ultracentrifuged (50,000 rpm, 1 hour) and washed five times using ultracentrifugation until no fluorescence was detected in the control microgel sample. The microgel was then resuspended in 2.5 mL Milli-Q water. The microgel coupled with Rhodamine B is referred to herein as RB-MG.

### Example 3

### Coupling of Streptavidin (SP) onto Microgel (MG)

The lyophilized microgel was resuspended in MES buffer (20 mM, pH 5.5) at a concentration of 2 mg/mL by mixing overnight. 1 mL microgel suspension was reacted with 40 μl streptavidin (1.22 mg/mL in Milli-Q water) in the presence of 100 mM EDC for 4 hours at room temperature. After the reaction, the microgel was ultracentrifuged (50,000 rpm, 50 mins) and washed twice using 2 mL MES buffer with stirring for 30 mins and ultracentrifugation as above (SP-MG). The pellet was then resuspended in 1 mL phosphate buffer (10 mM, pH 7.4). A control was done with the same procedure but without EDC (SP-MG-control). After washing, no protein could be detected by Bradford microassay either in the supernatants or in the suspension of the control sample. The amount of SP coupled on the microgel surface was determined to be 7.5 μg SP/(mg microgel) by analyzing the SP-MG suspension using Bradford microassay protocol.

### Example 4

### Immobilization of anti-rabbit IgG onto SP-Microgel

Ten μl anti-rabbit IgG biotin conjugate (Biotin-IgG) (0.5 mg/mL from Sigma) was incubated with 1 mL SP-MG suspension for 1 hour at room temperature. The microgel was then ultracentrifuged (50,000 rpm, 50 mins) and washed twice using 2 mL buffer (10 mM sodium phosphate, pH 7.4) with stirring for 30 mins, followed by ultracentrifugation as above. The pellet was then suspended in 1 mL phosphate buffer (10 mM, pH 7.4) (IgG-MG). A control was done as with the SP-MG-control by following the same procedure (IgG-MG-control). Also, after the sample was washed, no protein could be detected using the Bradford microassay in the supernatant and in the suspension of the control sample.

The antigen, anti-mouse IgG, used to determine the activity of the anti-rabbit IgG was developed in rabbit using purified mouse IgG as the immunogen. Therefore it was essentially rabbit IgG and was the antigen for the anti-rabbit IgG. Since the antigen was conjugated with peroxidase (AG-Per), so the activity of the anti-rabbit IgG on microgel surface can be determined using the substrate for peroxidase. Briefly, 5 μl anti-rabbit Ag-Per (8 mg/mL) was incubated with 1 mL IgG-MG in buffer (10 mM sodium phosphate, pH 7.4) for 1 hour at room temperature. After ultracentrifugation (50,000 rpm, 50 mins), it was washed twice by 2 mL buffer with 30 mins with stirring followed by ultracentrifugation as above, and then resuspension in 1 mL of buffer (Ag-MG). The same procedure was used as with the IgG-MG-control (Ag-MG-control). Again, washing was confirmed by Bradford microassay. Then, 1 μl Ag-MG suspension and Ag-MG-control suspension was incubated with 1 mL OPD solution, which is the substrate for the peroxidase, for half an hour and the absorbance at 450 nm was recorded using a UV-VIS spectrophotometer.

### Example 5

### Immobilization of ATP-Binding Aptamer onto SP-MG

Two μl (183 μM) biotin-aptamer-fluorescein was incubated with 1 mL SP-MG suspension prepared as described above at room temperature for 1 hour. The microgel was then ultracentrifuged and suspended in 1 mL of binding buffer (300 mM NaCl, 5 mM MgCl₂, 25 mM Tris-HCl, pH=8.3). A control was done with SP-MG-control sample. By checking with the fluorometer, there was no DNA aptamer adsorbed on the control microgel and all the aptamer was coupled on the SP-MG. Then the activity of the coupled aptamer on the microgel surface was determined. A 125 μl APT-MG was diluted to 1 mL using binding buffer. After the fluorescence signal became stable, 10 μL QDNA (10 μM) was introduced, then after the fluorescence signal became steady again, 10 μL ATP or GTP (100 mM) was added in to induce the specific binding.

### Example 6

### Titration

Simultaneous conductometric and potentiometric titration of the microgel was carried out by a Burivar-I2 automatic buret (ManTech Associates) at 25° C. to quantify the amount of carboxyl groups on the microgel surface. Briefly, 50 mg lyophilized microgel was resuspended in 50 mL 1 mM NaCl. Both a slow base-into-acid titration (67 min/unit pH) and a fast base-into-acid titration (6.7 min/unit pH) were conducted to get a repetition.

### Example 7

### Paper Chromatography

Whatman No. 1 filter paper was cut into rectangular pieces along the machine direction. In some cases filter paper strips were soaked in 0.1% PAE resin solution for 45 mins and then heated to 120° C. for 10 mins. Moreover, some PAE treated strips were subsequently soaked in 0.5% PAA (MW 30 KDa) or 0.5% CMC (MW 90 kDa, DS 0.7) for 30 mins and then let dry in the air. The paper strips for RB-MG and APT-MG were used with no further treatment, while the ones for IgG-MG were treated with 0.5 wt % bovine serum albumin (BSA) by soaking for 1 h and dried in the air.

One microliter of microgel solution was spotted on the paper surface using a micropipet to get a line across the paper strip 1.5 cm from the bottom and then allowed to air-dry. The papers were then eluted with different samples or buffers. The bottom of the paper strip was dipped into the buffer at a depth of about 1 cm. After elution the paper strips were dried in the air. The APT-MG sample was first quenched with the QDNA before being spotted on paper. For the RB-MG and APTMG samples, the fluorescence intensity of the paper strip was scanned using a Typhoon 9200, variable mode imager (Molecular Dynamics).

### Example 8

### Washing Experiment

1 μl Rhodamine B-labelled microgel solution was dropped on the filter paper strip (1 cm×3.5 cm) by a micropipette. Then the paper strip was put into 60 mL buffer and incubated for 30 mins with stirring. In order to introduce some wet strength, the filter paper was treated with PAE resin. Then some of them were treated with PAA or CMC as described above. Two continuous washes were done. The fluorescence intensity of the paper strip was scanned by Typhoon before and after each wash to check whether the microgel sticks on paper.

### Example 9

### Printing

The printing of microgels onto filter paper surface was performed by a Dimatix Materials Printer, DMP-2800 series (Fujifilm Dimatix, Inc., 2230 Martin Ave., Santa Clara, Calif.). The aptamer ink consisted of 0.67 mg/mL quenched aptamer-MG in binding buffer. The word “SU” was printed using a drop volume of 10 pL with 20 μm between neighboring drops. Five layers were printed for the quenched aptamer-MG.

### Example 10

### Fluorescence Labelling of Bovine Serum Albumin

250 μl FITC solution (1 mg/L in DMSO) was added to 5 mL BSA solution (2 mg/mL in 0.1 M sodium carbonate, pH=9) and allowed to react overnight at 4° C. in the dark (Hermanson, G. T.; Editor, Bioconjugate Techniques. 1995; p 786). The product was purified by passing through a Sephadex G-25 column and then freeze dried. The dried protein was redissolved in Milli-Q water at a concentration of 0.72 mg/mL tested by BCA reagent (Sigma).

### Example 11

### Electrophoretic Mobility

Electrophoretic mobility was measured by a ZetaPlus analyzer (Brookhaven Instruments Corp.) operating in phase analysis light scattering mode (PALS). Samples were dissolved in 1 mM sodium chloride as the background. A total of 10 runs (15 cycles each) were carried out for each sample.

### Example 12

### Dynamic Light Scattering

Particle sizes of the microgels were determined by dynamic light scattering with a detection angle of 90°. A Melles Griot HeNe laser was operated at 632.8 nm as the light source. The detector model was BI-APD. Correlation data were analyzed by BIC (Brookhaven Instruments Corp) dynamic light scattering software (9 kdlsw32 ver. 3.34) using the cumulants model. Microgels were suspended in filtered 1 mM NaCl and pH values were adjusted by 0.1 M HCl or 0.1 M NaOH. The scattering intensity was adjusted between 100 and 250 kilocounts/s. The duration time for each run was set up to 10 minutes and three replicates were conducted for each sample.

### Example 13

### Confocal Microscopy

Confocal microscopy was conducted with Zeiss LSM 510 laser scanning confocal microscope. A stack of images in the xy plane was taken though the z direction, from which xz cross sections were generated. The multi-track mode was used to check how the protein behaves in the microgel region.

### Results

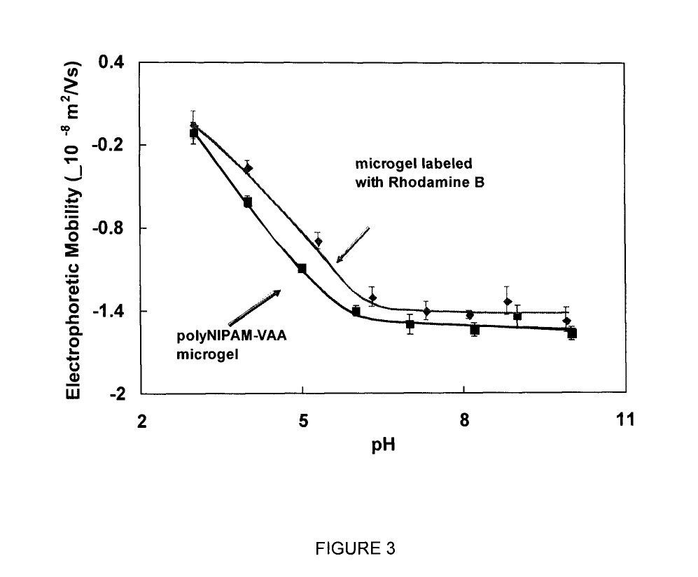

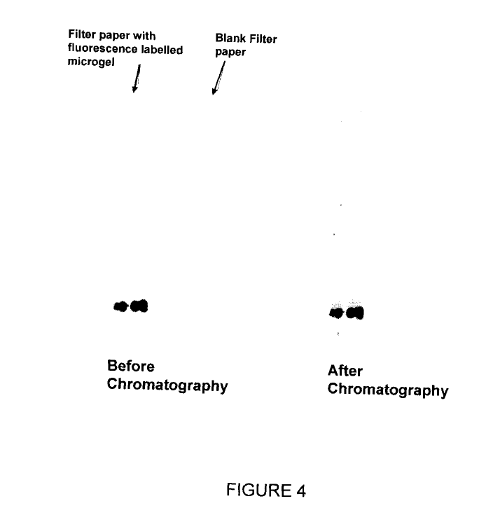

The microgel (MG) was prepared from a mixture of N-isopropylacrylamide (0.72 wt %), vinyl acetic acid (0.056 wt %) and N-methylenebisacrylamide (0.052 wt %) resulting in monodisperse particles with an average particle diameter of 275 nm under conditions of low swelling. From the titration results, the carboxylate content of microgel was determined to be 0.248±0.023 meq per gram of dry gel by conductometric titration. Previous work has shown that because vinyl acetic acid reacts by chain transfer, most of the carboxyl groups are located on chain ends on the microgel surface (Hoare, T.; Pelton, R. Macromolecules 2004, 37, 2544-2550; Hoare, T.; Pelton, R. Langmuir 2004, 20, 2123-2133). To facilitate detecting the migration of the microgel on the paper surface, the microgel was further derivatized with the red fluoroflore (Rhodamine B) giving the labelled microgel MG-RB. FIG. 3 shows that the microgel was still negatively charged after labeling with Rhodamine B and the pH dependence of their mobilities was quite the same, which indicates that this modification did not change the surface charge property of the microgel. In FIG. 4, the black line was the Rhodamine B labelled microgel deposited on filter paper strip by micropipette. The paper strip was developed in sodium phosphate buffer for about 10-15 mins. The paper strip before and after chromatography was scanned by Typhoon. In this Figure, it can be seen that the intensity of the black area almost does not change, which means that microgel does not move on filter paper surface in the chromatography experiment. A black line was observed at the top of both the strips which corresponds to the fluorescence background of the filter paper.

Furthermore, developing in buffer having two alternative pH's was utilized to study the effect of pH. In these experiments, paper strips were treated with PAE resin first, then with PAA (PAE-PAA paper) or CMC (PAE-CMC paper) as described above. PAE resin was used to introduce some wet strength to the filter paper. Treating the paper strips with PAA or CMC subsequently neutralized the positive charge of PAE resin. It was shown that changing the pH had no effect. Since many biochemistry experiments need high ionic strength and even need surfactants, such as SDS and Tween 20, paper chromatographies were also done at very high salt concentration with SDS or Tween 20. The results demonstrated that both high ionic strength and surfactant did not make the microgel migrate on filter paper.

It was concerned that the paper strips might not be long enough and the microgel would not have enough time to move, so a longer strip (2 cm×14 cm) was employed. The chromatography took much more time than the ones with shorter strips, and the microgel still did not move. So whether the microgel will migrate on paper does not depend on the developing time.

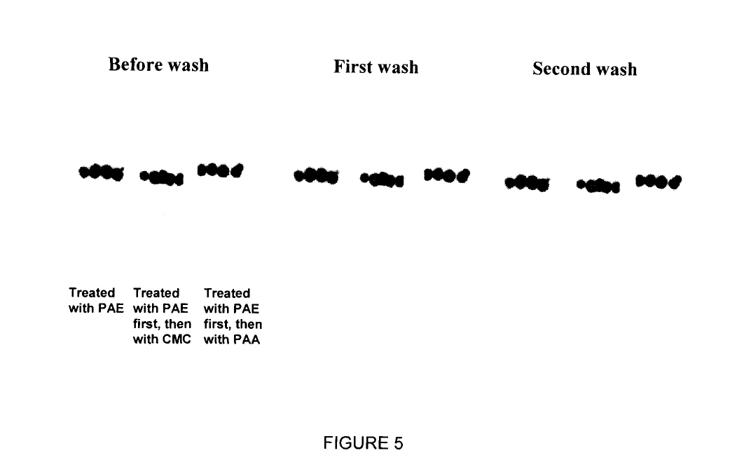

Washes of the paper strips were also performed to check whether the microgel would stick on paper in the incubation experiment. FIG. 5 indicates that after two continuous washes most of the microgel remained on the filter paper. Buffers with different pHs, ionic strengths, and/or detergent were also investigated and same result (not shown) was obtained as in FIG. 5. Experiments demonstrated that it was desirable to allow the spotted microgel to dry before performing elution experiments to improve fixing of the microgel to the paper fiber matrix.

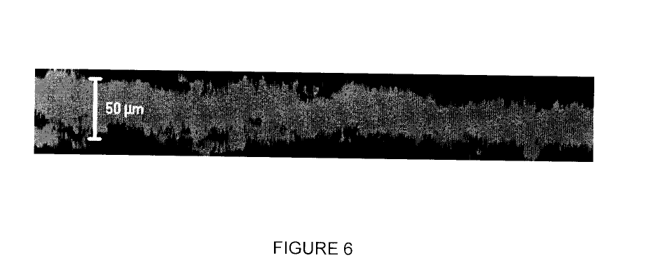

Laser scanning confocal microscopy was used to obtain the optical micrograph of the paper cross section on the band where the RG-MG was spotted. The picture in FIG. 6 was the projection of the stack images in the Z direction at the microgel area. The image shows that the microgel penetrated about 50 μm into the paper corresponding to about one third of the paper thickness. While not wishing to be limited by theory, it is proposed that capillary forces carry the particles deeply into the open paper structure. After drying, mechanical entrapment retards redipsersion and transport during the relatively quiescent chromatographic elution.

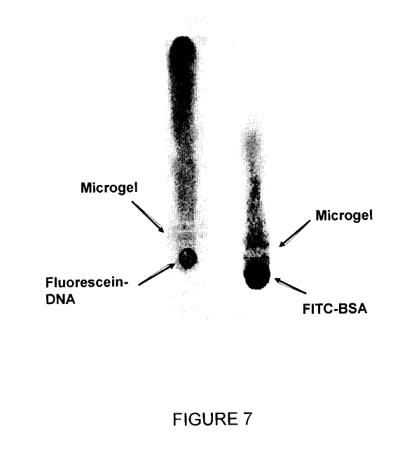

The final goal of this work was to use polyNIPAM microgel to support bioactive agents. The results above have already confirmed that the microgel sticks on filter paper and does not come off. However, in order to be applied to chromatography, the samples to be detected should be able to pass through the microgel region to let the specific detection reaction occur. In other words, the microgel should not block the migration of the sample on paper surface. The samples could be, for example, proteins, DNAs or small molecules. Since most likely, small molecules will migrate easier than proteins and DNAs, DNA oligo and BSA were studied as representatives for the samples. They were both fluorescently labelled to facilitate imaging their migration on filter paper. In FIG. 7, it can be seen that both the BSA and the DNA oligo passed through the microgel region. Moreover, this figure also shows that the DNA oligo moved better than the protein. In addition, confocal microscopy was employed to check how the BSA distributed at the microgel region after the chromatography described above. In these experiments, it was clearly demonstrated that BSA and microgel have molecular scale contact. This allows the detection reaction to happen. Also, it was again shown that the microgel did not move on the paper at all.

In order to reduce the non-specific binding of proteins with paper, the blank filter paper strips were treated with either defatted milk or bovine serum albumin (BSA). First, whether the microgel will move on protein treated paper surface was checked and it was found that microgel did not move. Then chromatographies were developed in FITC-BSA solutions to check how samples migrate on the paper strips treated with or without proteins. BSA moved much better on the paper stripes treated either with milk protein or BSA.

In addition, for the incubation experiment, it was determined whether the DNA or protein have non-specific binding with the filter paper. To do this, experiments were conducted to see of these samples could be washed off the paper surface. DNA or protein was dropped onto the filter paper surface first, and then after it was dry, the paper strips were continuously washed. It was found that most of the DNA oligos could be washed off while protein could not.

Streptavidin was coupled to the pNIPAM-VAA carboxylated microgels which were decorated with either antibodies or DNA aptamers (see FIG. 1). Streptavidin-coupled microgel (SP-MG) was prepared with a streptavidin content of 7.5 μg per mg of dry microgel (i.e. 0.75 wt %)—see FIG. 1. Assuming all of the streptavidin was located on the exterior surfaces of the microgels and that the water content was 45% (low swelling conditions), 7.5 μg of protein per mg of dry microgel corresponds to a coverage of ˜0.2 mg/m² which is an order of magnitude less that the 5 mg/m² value for streptavidin physical adsorption on to polystyrene latex reported by Caldwell (Huang, S. C.; Swerdlow, H.; Caldwell, K. D. Analytical Biochemistry 1994, 222, 441-449). In addition, a control sample, SP-MG-control, was prepared by same procedure except the EDC coupling catalyst was not added.

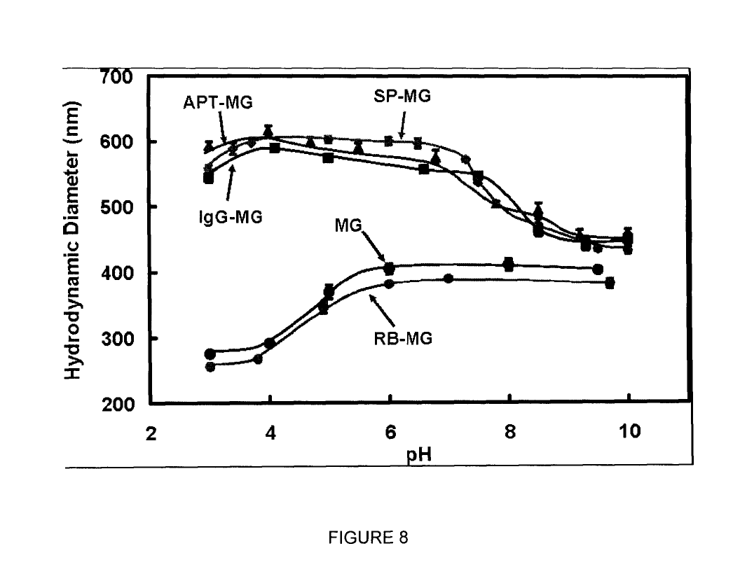

Microgel-supported DNA aptamer (APT-MG) was prepared by treating SP-MG with a biotinylated aptamer which recognizes ATP (Nutiu, R.; Li, Y. F. J. Am. Chem. Soc. 2003, 125, 4771-4778; Nutiu, R.; Li, Y. F. Angew. Chem. In. Ed. 2005, 44, 1061-1065). Similarly, microgel-supported IgG (IG-MG) was prepared by treating SP-MG with anti-rabbit IgG biotin conjugate. The hydrodynamic diameters of the 4 microgels (RB-MG, SP-MG, APT-MG and IG-MG) were determined as a functions of pH and the results are summarized in FIG. 8. The diameter of the starting MG increased with pH reflecting the donnan contribution to swelling with ionization of the carboxyl groups (Hoare, T.; Pelton, R. Macromolecules 2004, 37, 2544-2550). Streptavidin coupling to give SP-MG had a profound effect on the particle size. At low pH the size doubled compared to MG. Furthermore, increasing pH caused a decrease in SP-microgel size which is opposite to the behavior of MG. The antibody and aptamer modifications had little influence on the microgel swelling.

The streptavidin modification increased the particle diameter by a factor of 1.5 at neutral pH. The streptavidin content of ST-MG was 7.5 μg per mg of dry microgel. This cannot account for the doubling of gel diameter by particle growth and swelling. While not wishing to be limited by theory, an explanation is that the streptavidin coupling induced limited aggregation of the microgels. The coupling was performed at pH 5.5 where the streptavidin is slightly positively charged (Leckband, D. E.; Schmitt, F. J.; Israelachvili, J. N.; Knoll, W. Biochemistry 1994, 33, 4611-4624; van Oss, C. J.; Giese, R. F.; Bronson, P. M.; Docoslis, A.; Edwards, P.; Ruyechan, W. T. Colloids and Surfaces B-Biointerfaces 2003, 30, 25-36) which would favor limited flocculation of the anionic microgels. Using a two-step CDC-NHS coupling will allow washing the activated microgel before introducing steptavidin

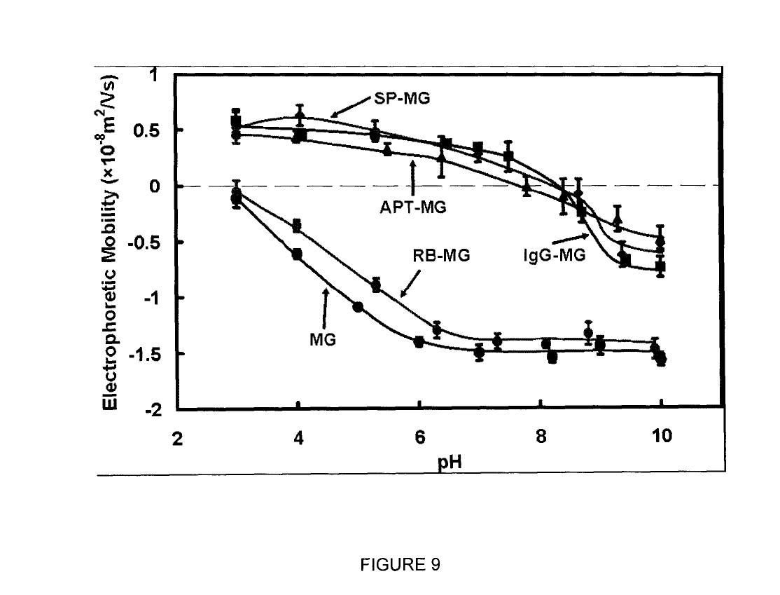

The electrophoretic mobilities of the four microgels are shown as functions of pH in FIG. 9. The polyNIPAM-VAA microgel has low negative, pH dependent mobility reflecting a swollen state with surface localized carboxyl groups (Hoare, T.; Pelton, R. Polymer 2005, 46, 1139-1150). The streptavidin-modified gels were slightly positive up to pH 8 beyond which they were slightly negative. Aptamer and IGT modification of the SP-microgel did not influence the electrophoresis very much.

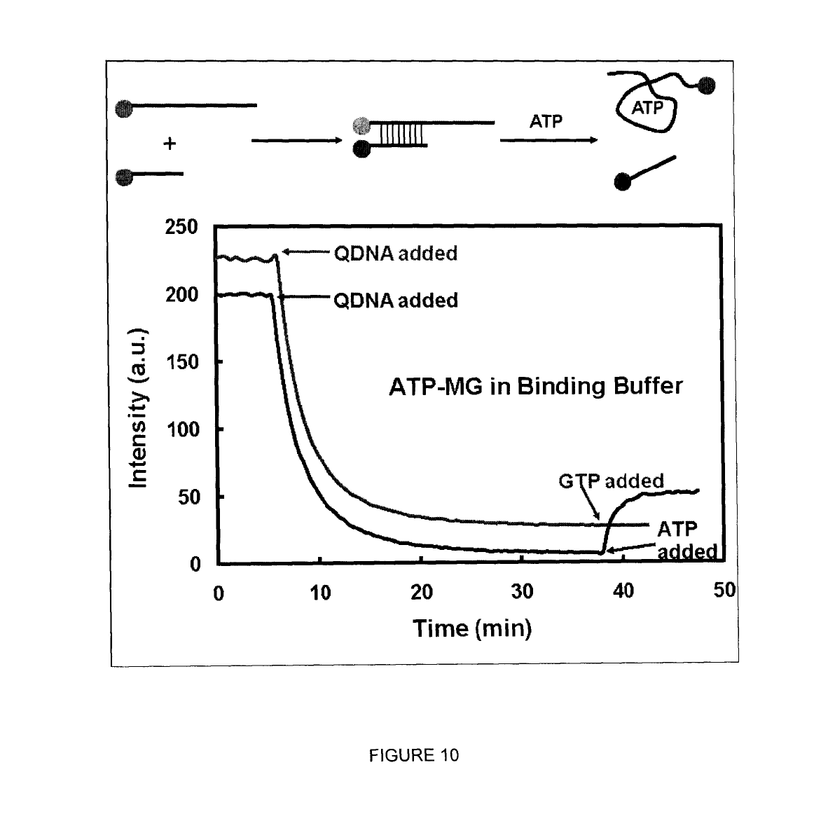

As mentioned above, the ATP-MG comprised an aptamer that specifically binds to ATP. The activity was measured using the structure-switching method (Su, S.; Nutiu, R.; Filipe, C. D. M.; Li, Y.; Pelton, R. Langmuir 2007, 23, 1300-1302). In this approach, illustrated in FIG. 10, a duplex is made from an aptamer sequence with a fluorescent terminus and an antisense oligonucleotide for the aptamer endcapped with a fluorescent quencher. The sensor is first activated by forming a duplex with the quencher terminated antisense oligonucleotide (QDNA) which locates a quencher close to the fluorescent group on the aptamer. The fluorescence intensity of the duplex is low because of quenching. Exposure of the duplex to ATP in binding buffer causes increased fluorescence because the duplex dissociates and ATP binds to aptamer, separating the fluorescent group from the quencher. Note that this particular aptamer is not optimized for physiological conditions. Instead, the “binding buffer” (300 mM NaCl, 5 mM MgCl₂, 25 mM Tris-HCl, pH) 8.3) contains metal ions that facilitate aptamer folding in the presence of ATP.

The functionality of the APT-MG in solution was evaluated using the scheme illustrated in FIG. 10 and the results are also shown in FIG. 10. The lower curve shows fluorescence as a function of time for APT-MG. The high initial fluorescence plummeted upon addition of the quencher terminated anti-sense oligonucleotide (QDNA) because duplex formation placed the quencher near to the fluorescent terminus of the aptamer (see FIG. 10). Subsequent addition of ATP displaced some of the bound QDNA giving a rise in fluorescence. The specificity of the DNA aptamer is illustrated by the upper curve which shows that GPT addition does displace the QDNA to re-activate the fluorescence. The conclusion from FIG. 10 is that the DNA aptamer could detect ATP in spite of being attached to the microgel.

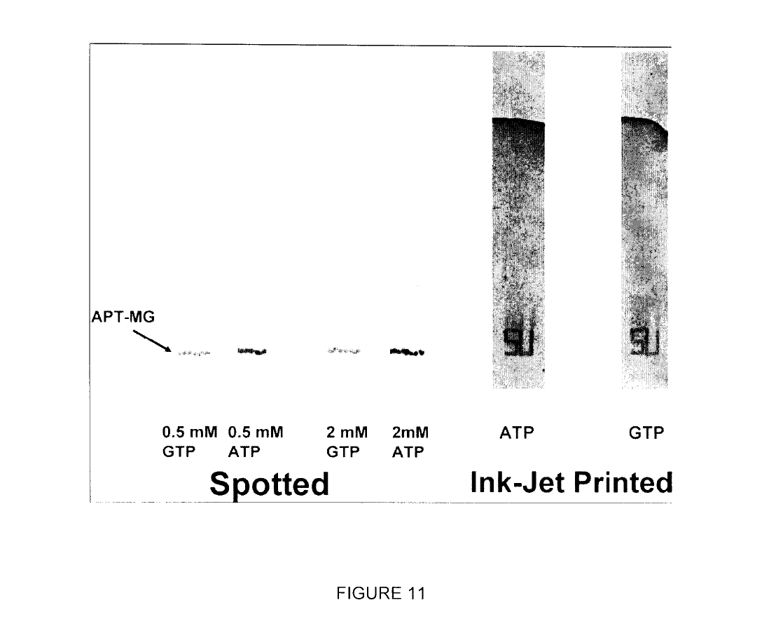

One goal of the present research was to demonstrate the activity of the APT-MG on paper surfaces. For this 1 μl aliquots (6.5 mg/mL) of microgel (quenched with QDNA in advance) were spotted or printed as a band on filter paper strips giving coverage of approximately 3.25×10⁻² mg of dry microgel per m² of paper. After room temperature drying, the paper strips were eluted with either ATP or GTP in binding buffer at pH 8.3 and the strips were scanned. FIG. 11 shows the strips after elution with ATP or GTP in binding buffer, followed by room temperatures and drying. The bands at the bottom of the strips were the microgels, which, as shown above, do not migrate. The fluorescing microgels appear as black bands in these monochrome images. The microgels exposed to ATP gave greater fluorescence than the GTP control. This result shows not only that the APT-MG is active on the paper surface but also that ATP infiltrates the microgels during the elution.

A particular advantage of the microgel-supported biosensors is that they are small, uniform and robust which means that they can be formulated into coatings and inks. This was illustrated by printing the microgels with a Fuji-Dimatix Materials inkjet printer (DMP-2800 Series). FIG. 11 also shows two test strips, one eluted with ATP in buffer and the other with GTP. The microgels were printed forming the letters“SU” and, as with the spotted gel results in FIG. 11, the ATP eluted strip showed a much darker image indicating selective binding of ATP on the AT-MG gels.

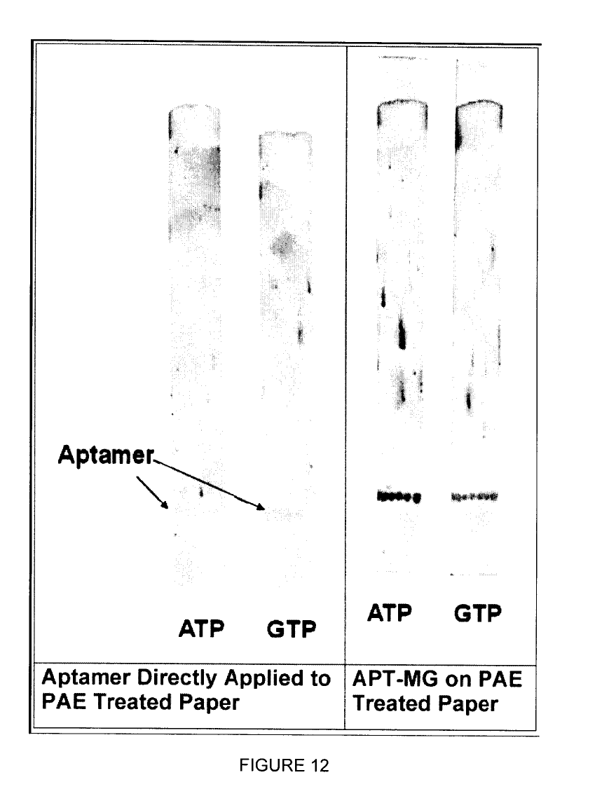

Another objective in using microgels was both to avoid the direct coupling of aptamers to paper and to protect the aptamers from hostile paper surfaces. To illustrated the second point, Whatman no. 1 filter paper was saturated with 0.1% commercial polyamide-amine-epichlorohydrin (PAE) wet strength resin, dried, and cured at 120° C. for 10 min to give a cationic surface on cellulose (Espy H. H. Tappi J. 195, 78, 90-99). The left-hand image in FIG. 12 shows strips where the aptamer solution was directly applied and then eluted with ATP or GTP. The directly applied aptamer seems to be nonfunctional on the PAE-paper surface as no significant fluorescence enhancement was observed with the ATP elution over the GTP elution (in fact, the GTP elution produced a slightly stronger signal). In contrast, the right-hand image in FIG. 12 shows APT-MG on PAE-treated paper. Once again, the sample eluted with ATP gave a much darker (more fluorescence) strip than the GTP eluted strip.

To illustrate the general utility of microgels as a biosensor support platform, microgels were prepared with anti-rabbit IgG, IgG-MG. The activity of IgG-MG was evaluated by exposing the micro gel particles to the antigen (anti-mouse peroxidase conjugate, Ag-Per), removing the excess antigen in the serum by centrifugation and re-dispersion. The antigen content of the cleaned gels was determined by exposing the sample to o-phenylenediamine dihydrochloride (OPD) and measuring the absorption at 450 nm. The color change was catalyzed by the peroxidase enzyme which was conjugated to the Ag-Per antigen. The absorption from the IgG-MG was nine times higher than the absorption from the IgG-MG-control (which was prepared without EDC as the coupling agent). This result illustrates that the IgG-MG microgels are very hydrophilic and have little non-specific affinity for proteins.

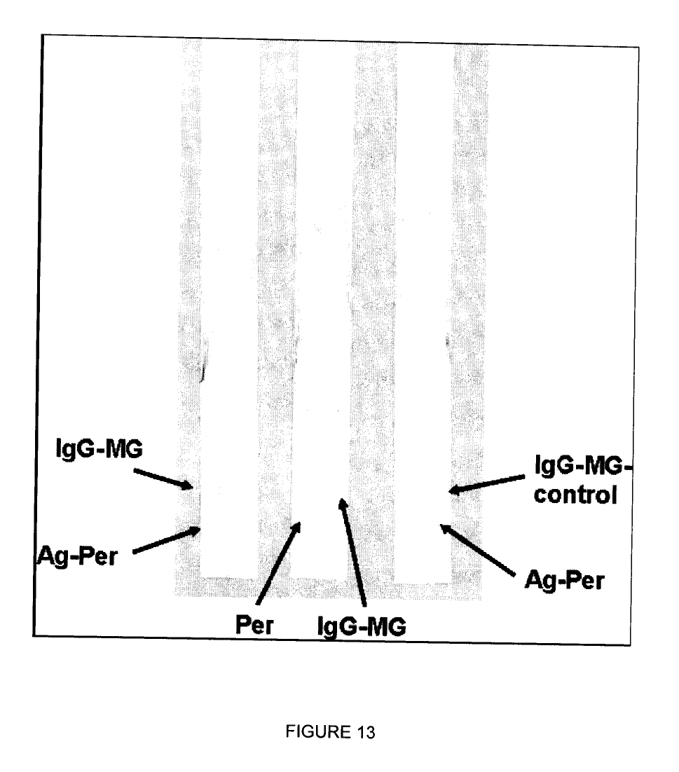

Two procedures were developed to illustrate the activity of IgG-MG on paper and the details are summarized in Table 1. For procedure 1 the antibody was spotted on the paper below the IgG-MG whereas for procedure 2 the antibody was present in the eluting solution. The results for procedure 1 are summarized in FIG. 13. A yellow color on the strips was from the peroxidase-catalyzed oxidation of OPD. The left-hand strip shows an intense band corresponding to the location of immobilized IgG-MG. Two elution steps were required to achieve this band. In the first, the antigen (Ag-Per) was carried past the microgel by the buffer. In the second step, after drying, the OPD was eluted up the paper strip. Therefore the presence of a band at the microgel indicates specific binding of the Ag-Per and that the IgG-MG was active. The middle strip in FIG. 13 shows a control experiment that employed peroxidase alone, instead of the antigen-peroxidase conjugate. The absence of a band in the microgel region confirms that there was no binding of peroxidase with the microgel. Finally, the right-hand strip in FIG. 13 shows the result for the IgG-MG-control. This microgel was prepared by the following steps: (1) the microgel was mixed with streptavidin but without EDC as the coupling agent, and the product was washed to produce SP-MG-control; (2) the SP-MG-control was treated with Ag-Per and washed to produce IgG-MG-control. The absence of a band in the right-hand strip confirms the absence of nonspecific binding to the microgel.

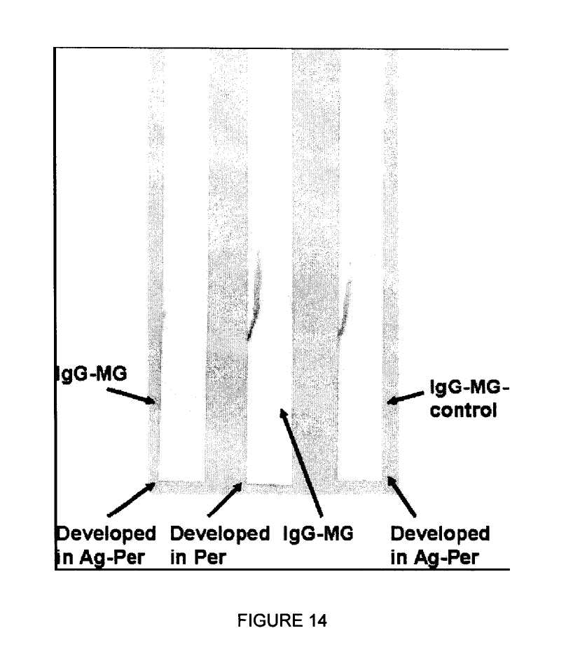

In the second procedure the antigen was not spotted on the paper but instead was eluted from solution. The results are summarized in FIG. 14. As before, the left-hand strip confirms the activity of IgG-MG and the other two control strips confirm the absence of nonspecific bonding to microgel.

### Discussion

polyNIPAM microgel was deposited onto a filter paper surface by directly dropping with micropipette. For chromatography, blank filter paper was good enough to hold the microgel and there was no need to treat the paper with polymers. For the incubation approach, paper strips were treated with PAE resin first to give them the wet strength. Since PAE will make the paper positively charged, they were further treated with PAA or CMC. The microgel did not come off the filter paper after two continuous washes. Moreover, these results did not depend on the ionic strength, pH values and the presence of detergent. These results mean that the microgels have a great potential to be used as a detection support on paper surfaces under a variety of reaction conditions.

In FIG. 7, it was shown that the microgel did not block the migration of both the DNA and protein on filter paper in chromatography. This is beneficial because the sample must pass through the microgel to let the detection reaction occur. More importantly, the sample should be able to contact the biodetective agents coupled on the microgel at the molecular level, and this has been demonstrated. It also can be seen in FIG. 7 that DNA oligos move better than proteins. This is not surprising because the DNA oligos were very negatively charged while the proteins had many functional groups (positive, negative, or hydrophobic). Subsequently, milk protein or BSA was used to block the cellulose surface. This is a general step in an ELISA test. FIG. 8 shows that BSA moves much better on the paper surface after the paper surface was treated. This indicates that treating the filter paper with BSA or milk protein can reduce the non-specific interactions of the paper surface with proteins.

For the incubation approach, FIG. 9 shows that most of the DNA oligos could be washed off after the paper strip was treated with a negatively charged polymer. However, in FIG. 10, BSA could not be washed off from the paper strips even after treatment with milk protein.

### (K) Conclusions

Since Martin and Synge's Nobel prize 1952 for paper chromatography, cellulosic fiber surfaces have been widely used for separations and as supports in lateral flow or “dipstick” like biosensor applications including small molecules, proteins, oligonucleotides, and pathogens (Klewitz, T.; Gessler, F.; Beer, H.; Pflanz, K.; Scheper, T. Sensors and Actuators B-Chemical 2006, 113, 582-589; Xu, C.; Wang, H.; Peng, C.; Jin, Z.; Liu, L. Biomedical Chromatography 2006, 20, 1390-1394; Shim, W. B.; Yang, Z. Y.; Kim, J. Y.; Choi, J. G.; Je, J. H.; Kang, S. J.; Kolosova, A. Y.; Eremin, S. A.; Chung, D. H. Journal of Agricultural and Food Chemistry 2006, 54, 9728-9734; Liu, J. W.; Mazumdar, D.; Lu, Y. Angewandte Chemie-International Edition 2006, 45, 7955-7959; Renuart, I.; Mertens, P.; Leclipteux, T.; (Coris Bioconcept Sprl, Belg.). Application: WO, 2004, p 36; Smits, H. L.; Eapen, C. K.; Sugathan, S.; Kuriakose, M.; Gasem, M. H.; Yersin, C.; Sasaki, D.; Pujianto, B.; Vestering, M.; Abdoel, T. H.; Gussenhoven, G. C. Clinical and Diagnostic Laboratory Immunology 2001, 8, 166-169; Johnston, S. P.; Ballard, M. M.; Beach, M. J.; Causer, L.; Wilkins, P. P. Journal of Clinical Microbiology 2003, 41, 623-626; Ketema, F.; Zeh, C.; Edelman, D. C.; Saville, R.; Constantine, N. T. Journal of Acquired Immune Deficiency Syndromes 2001, 27, 63-70; Barrett, C.; Good, C.; Moore, C. Forensic Science International 2001, 122, 163-166; Leclipteux, T.; Degallaix, S.; Denorme, L.; Mertens, P.; Olungu, C.; (Coris Bioconcept S.P.R.L., Belg.). Application: EP, 2006, p 22; Saito, N.; Taya, T.; (Sysmex Corporation, Japan). Application: US, 2004, p 12; Nutiu, R.; Li, Y. F. Angewandte Chemie-International Edition 2005, 44, 1061-1065). Virtually all of these implementations either involved pure cellulose based chromatography paper or nitrocellulose films. In this work, it has been shown that microgels, large enough to isolate the biosensors from the paper surface, are sufficiently hydrophilic to be wetted during chromatographic elution, exposing the gel supported biosensors to their targets.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1
Procedures to illustrate activity of IgG-MG on paper.

| STEP | PROCEDURE 1 DESCRIPTION (FIG. 8) |
|---|---|
| 1 | Soak paper strip (1 × 10 cm) in 0.5 wt % BSA in water for 1 hour followed |
| | by air drying |
| 2 | 1 μL of microgel (or control) 2 or 10 mg/mL spotted in a band 15 mm |
| | from the bottom of the paper strip. |
| 3 | Spot 1 μl Ag-Per or Per solution in a band 10 mm from the bottom; their |
| | concentrations were: 8 mg/mL, 0.8 mg/mL, or 0.08 mg/mL. |
| 3 | Elute with buffer (10 mM sodium phosphate, pH 7.4, 0.5 wt % BSA, |
| | 0.05 wt % Tween 20) for 1 hour, then air dry |
| 4 | Elute with OPD solution (0.4 mg/mL in buffer, buffer prepared |
| | according to Sigma assay) for 30 minutes and air dry. |
| STEP | PROCEDURE 2 DESCRIPTION (FIG. 9) |
| 1 | Soak paper strip in 0.5 wt % BSA in water for 1 hour followed by air |
| | drying |
| 2 | 1 μL of microgel 10 mg/L spotted in a band 15 mm from the bottom and |
| | air dry |
| 3 | Elute with Ag-Per or Per solution (10 mM sodium phosphate, pH 7.4, |
| | 0.5 wt % BSA, 0.05 wt % Tween 20) for 1 hour, then air dry; their were |
| | 1.6 μg/mL |
| 4 | Elute with buffer (10 mM sodium phosphate, pH 7.4, 0.5 wt % BSA, |
| | 0.05 wt % Tween 20) for 1 hour, then air dry |
| 5 | Perform chromatography in OPD solution (0.4 mg/mL in buffer, buffer |
| | prepared according to Sigma assay) for 30 minutes and air dry |

Three strips were prepared for every experiment: 1) IgG-MG with Ag-Per; 2) IgG-MG with Per; and, 3) IgG-MG-control with Ag-Per

1. A method for attaching bioactive agents to paper products comprising contacting the paper with a solution comprising colloidal support particles under conditions for the immobilization of the particles to the paper, where the bioactive agents are immobilized on the colloidal support particles.

2. The method according to claim 1, wherein the colloidal support particles are microgels prepared from starch, cross-linked poly(sodium methylacrylate), poly(N-acryloylpyrrolidine), poly(N-acryloylpiperidine), poly(N-vinylisobutyramide), gums, functionalized latex, agarose or functionalized poly(N-alkylacrylamides) or poly(N,N-dialkylacrylamides).

3. The method according to claim 1, wherein the colloidal support particles comprise a functional group at or near their surface for immobilization of the bioactive agents.

4. The method according to claim 3, wherein the functional group is selected from a carboxyl, amino, thiol, aldehyde, cyano, hydroxyl, tosyl and hydrazine group.

5. The method according to claim 4, wherein the functional group is selected from a carboxyl and an amino group.

6. The method according to claim 4, wherein the colloidal support particles are poly(N-alkylacrylamide) or poly(N,N-dialkylacrylamide) microgels.

7. The method according to claim 1, wherein the colloidal support particles are carboxylated poly(N-alkylacrylamide) or poly(N,N-dialkylacrylamide) microgels

8. The method according to claim 6, wherein the N-alkylacrylamide or N,N-dialkylacrylamide is selected from N-isopropylacrylamide, N-ethylmethyacrylamide, N-n-propylacrylamide, N-methyl-N-n-propylacrylamide, N-isopropylmethylacrylamide, N-ethylacrylamide, N,N-diethylacrylamide, N-n-propylmethylacrylamide, N-cyclopropylacrylamide and N-methylacrylamide.

9. The method according to claim 8, wherein the N-alkylacrylamide is N-isopropylacrylamide.

10. The method according to claim 1, wherein the colloidal support particles are carboxylated poly(N-isopropylacrylamide) microgel prepared by copolymerization of N-isopropylacrylamide with vinyl acetic acid (VAA).

11. The method according to claim 1, wherein the contacting of the paper with a solution comprising the colloidal support particles is done using a micropipette or by deposition using any printing technique.

12. The method according to claim 1, wherein the conditions for the immobilization of the microgels to the paper comprise drying the paper after contacting with the microgel solution.

13. The method according to claim 1, wherein the paper is treated prior to contact with the microgel solution.

14. The method according to claim 13, wherein the paper is treated to minimize non-specific binding, to increase the paper wet strength or to neutralize charges on the paper or other pre-treatment.

15. The method according to claim 1, wherein the bioactive agent is selected from proteins, polypeptides, polynucleotides (DNA or RNA), nucleotide fragments, carbohydrates, other polymeric species, cage compounds and small inorganic or organic molecules.

16. The method according to claim 15, wherein the bioactive agent is selected from antibodies, antibody fragments, probes, primers, enzymes, catalysts, drugs, chelating agents and biotin.

17. Bioactive paper prepared using the method according to claim 1.

18. A method of detecting a target substance comprising contacting a solution or gas suspected of containing the substance with the bioactive paper according to claim 15 and observing a detectable change in an area on the paper where a bioactive agent has been deposited.

19. The method according to claim 18, wherein the detectable change is a change in the color, fluorescence, ultraviolet or infrared properties of the bioactive agent and/or substance.

20. The method according to claim 18, wherein the substance is a molecular species, cell or organism.

21. The method according to claim 20, wherein the substance is a pathogen or a toxic substance.
